(19) United States
(12) Patent Application Publication
(10) Pub. No.: US 2006/0030769 A1
(43) Pub. Date: Feb. 9, 2006

(54) System and method for loading timepoints for analysis of disease progression or response to therapy

(76) Inventors: Venkat Raghavan Ramamurthy, Malvern, PA (US); Arun Krishnan, Exton, PA (US); Christian Beldinger, Algonquin, IL (US); Juergen Soldner, Knoxville, TN (US); Maxim Mamin, Erlangen (DE); Axel Barth, Erlangen (DE); Stefan Käpplinger, Jena (DE); Michael Gluth, McHenry, IL (US); Peggy Hawman, Schaumburg, IL (US); Darrell Burckhardt, Hoffman Estates, IL (US); Axel Platz, Munchen (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/146,961

(22) Filed: Jun. 7, 2005

Related U.S. Application Data
(60) Provisional application No. 60/581,136, filed on Jun. 18, 2004.

(51) Int. Cl. A61B 5/05

(52) U.S. Cl. 600/407

(57) ABSTRACT

A system and method for loading a timepoint for comparison with a previously loaded timepoint are provided. The method comprises: selecting an image dataset of the timepoint; validating the image dataset of the timepoint against a validated image dataset of the previously loaded timepoint; and constructing a volume based on the image dataset of the timepoint.

### CROSS-REFERENCE TO RELATED APPLICATION

This application claims the benefit of U.S. Provisional Application No. 60/581,136, filed Jun. 18, 2004, a copy of which is herein incorporated by reference.

### BACKGROUND OF THE INVENTION

1. Technical Field

The present invention relates to medical image analysis, and more particularly, to a system and method for loading timepoints for analysis of disease progression or response to therapy.

2. Discussion of the Related Art

Functional imaging using single photon emission computed tomography (SPECT) and positron emission tomography (PET) is extremely valuable in the diagnosis of various medical disorders. Uncertainty in the anatomic definition on SPECT and PET images, however, sometimes limits their usefulness. To overcome this, a combination of magnetic resonance images (MRI) and X-ray computed tomography (CT) images with functional SPECT or PET images of the same sections of the body is sometimes used. This provides complementary anatomic (MRI or CT) and physiological (SPECT or PET) information that is of great importance to research, diagnosis and treatment.

Functional body images and structural images are two types of medical images used by medical practitioners for the diagnosis of certain medical disorders. Functional body images such as those derived from SPECT or PET scans, provide physiological information, whereas structural images such as those derived from CT or MRI, provide an anatomic map of the body. Different medical imaging techniques may provide scans with complementary and occasionally conflicting information. For example, the combination of such images (via image fusion or image registration) using picture archiving communications systems (PACS) can often lead to additional clinical information not apparent in the separate images. Thus, by imposing a structural anatomic framework on a set of functional images, the position of a tumor or other lesion in a later functional image may be determined even where there is insufficient anatomic detail.

Although the construction of a composite, overlapping medical image with image registration has been primarily used in the fusion of functional and anatomical images, it has also been applied to a series of the same modality of images. Examples of this are registration of SPECT images of the same subject in follow-up studies or in a comparison of an image with normal uptake properties to an image with suspected abnormalities. In addition, image registration of SPECT and PET images and the registration of SPECT and PET images with anatomic atlases provide an important means to evaluate comparative uptake properties of SPECT and PET radiopharmaceuticals, and to correlate uptake properties with anatomy.

Multi-modal medical image registration is fast becoming a visualization tool that can significantly aid in the early detection of tumors and other diseases and aid in improving the accuracy of diagnosis. For example, radiologists often have difficulty locating and accurately identifying cancer tissue, even with the aid of structural information such as CT and MRI because of the low contrast between the cancer and the surrounding tissues in CT and MRI images. However, by using SPECT and radioactively labeled monoclonal antibodies it is possible to obtain high contrast images of the concentration of antibodies in tumors.

It is thus becoming increasingly desirable to combine the output and strengths of multiple medical imaging systems. However, certain drawbacks exist due to combining different file structures, the transfer and networking thereof and registration and visualization of the composite images. For example, such systems typically do not support more than a few combinations of datasets from different modalities. In addition, many systems do not provide a quick and accurate means for analyzing changes in tumors. Further, many systems do not provide a quick technique for aligning medical images from different timepoints. For example, to accurately analyze changes in tumors, it is often necessary to compare images of the same modality that were scanned at different timepoints.

Accordingly, there is a need for a technique that enables medical practitioners to compare patient scans taken at a different times using the same or different modalities so that medical practitioners can make better-informed diagnostic, therapy and follow-up decisions in a cost-effective and efficient manner.

### SUMMARY OF THE INVENTION

The present invention overcomes the foregoing and other problems encountered in the known teachings by providing a system and method for loading timepoints for analysis of disease progression or response to therapy.

In one embodiment of the present invention, a method for loading a timepoint for comparison with a previously loaded timepoint, comprises: selecting an image dataset of the timepoint; validating the image dataset of the timepoint against a validated image dataset of the previously loaded timepoint; and constructing a volume based on the image dataset of the timepoint.

The image dataset of the timepoint and an image dataset of the previously loaded timepoint each comprise data acquired from one of a computed tomography (CT), positron emission tomography (PET), single photon emission computed tomography (SPECT), magnetic resonance (MR) and ultrasound modality.

The image dataset of the timepoint and the image dataset of the previously loaded timepoint each comprise one of a CT image series and MR image series, a PET image series and SPECT image series, a combination of a CT and PET image series, a combination of an MR and PET image series, a combination of a CT and SPECT image series, a combination of an MR and SPECT image series and an ultrasound image series.

The image series in each of the image dataset of the timepoint and the image dataset of the previously loaded timepoint comprise data from one of a pre-therapy, ongoing therapy and post-therapy study.

The method further comprises: registering the image dataset of the timepoint and the image dataset of the previously loaded timepoint using one of automatic registration, landmark registration and visual registration. The automatic registration used during the step of registering the image dataset of the timepoint and the image dataset of the previously loaded timepoint comprises: registering a first image series with a second image series of the image dataset of the timepoint; registering the first image series of the image dataset of the timepoint with a first image series of the image dataset of the previously loaded timepoint; and registering the first image series of the image dataset of the previously loaded timepoint with a second image series of the image dataset of the previously loaded timepoint.

In another embodiment of the present invention, a method for loading timepoints for analysis of disease progression or response to therapy, comprises: selecting an image dataset of a first timepoint; loading the image dataset of the first timepoint; validating the image dataset of the first timepoint; constructing a volume based on the image dataset of the first timepoint; selecting an image dataset of the second timepoint; loading the image dataset of the second timepoint; validating the image dataset of the second timepoint against the validated image dataset of the first timepoint; and constructing a volume based on the image dataset of the second timepoint.

The image dataset of the first timepoint and an image dataset of the second timepoint each comprise data acquired from one of a computed tomography (CT), positron emission tomography (PET), single photon emission computed tomography (SPECT), magnetic resonance (MR) and ultrasound modality.

The image dataset of the first timepoint and the image dataset of the second timepoint each comprise one of a CT image series and MR image series, a PET image series and SPECT image series, a combination of a CT and PET image series, a combination of an MR and PET image series, a combination of a CT and SPECT image series, a combination of an MR and SPECT image series and an ultrasound image series.

The image series in each of the image dataset of the first timepoint and the image dataset of the second timepoint comprise data from one of a pre-therapy, ongoing therapy and post-therapy study.

The method further comprises: determining whether the image dataset of the first timepoint was ambiguously selected; and determining whether the image dataset of the second timepoint was ambiguously selected.

In yet another embodiment of the present invention, a system for loading a timepoint for comparison with a previously loaded timepoint, comprises: a memory device for storing a program; a processor in communication with the memory device, the processor operative with the program to: select an image dataset of the timepoint; validate the image dataset of the timepoint against a validated image dataset of the previously loaded timepoint; and construct a volume based on the image dataset of the timepoint.

The image dataset of the timepoint and an image dataset of the previously loaded timepoint each comprise data acquired from one of a computed tomography (CT), positron emission tomography (PET), single photon emission computed tomography (SPECT), magnetic resonance (MR) and ultrasound modality.

The image dataset of the timepoint and the image dataset of the previously loaded timepoint each comprise one of a CT image series and MR image series, a PET image series and SPECT image series, a combination of a CT and PET image series, a combination of an MR and PET image series, a combination of a CT and SPECT image series, a combination of an MR and SPECT image series and an ultrasound image series.

The image series in each of the image dataset of the timepoint and the image dataset of the previously loaded timepoint comprise data from one of a pre-therapy, ongoing therapy and post-therapy study.

The processor is further operative with the program code to register the image dataset of the timepoint and the image dataset of the previously loaded timepoint using one of automatic registration, landmark registration and visual registration. The processor is further operative with the program code when automatically registering the image dataset of the timepoint and the image dataset of the previously loaded timepoint to: register a first image series with a second image series of the image dataset of the timepoint; register the first image series of the image dataset of the timepoint with a first image series of the image dataset of the previously loaded timepoint; and register the first image series of the image dataset of the previously loaded timepoint with a second image series of the image dataset of the previously loaded timepoint.

In an embodiment of the present invention, a system for loading timepoints for analysis of disease progression or response to therapy, comprises: a memory device for storing a program; a processor in communication with the memory device, the processor operative with the program to: select an image dataset of a first timepoint; load the image dataset of the first timepoint; validate the image dataset of the first timepoint; construct a volume based on the image dataset of the first timepoint; select an image dataset of the second timepoint; load the image dataset of the second timepoint; validate the image dataset of the second timepoint against the validated image dataset of the first timepoint; and construct a volume based on the image dataset of the second timepoint.

The image dataset of the timepoint and an image dataset of the previously loaded timepoint each comprise data acquired from at least one of a computed tomography (CT), positron emission tomography (PET), single photon emission computed tomography (SPECT), magnetic resonance (MR) and ultrasound modality.

The image dataset of the first timepoint and the image dataset of the second timepoint each comprise one of a CT image series and MR image series, a PET image series and SPECT image series, a combination of a CT and PET image series, a combination of an MR and PET image series, a combination of a CT and SPECT image series, a combination of an MR and SPECT image series and an ultrasound image series.

The image series in each of the image dataset of the first timepoint and the image dataset of the second timepoint comprise data from one of a pre-therapy, ongoing therapy and post-therapy study.

The processor is further operative with the program code to: determine whether the image dataset of the first timepoint was ambiguously selected; and determine whether the image dataset of the second timepoint was ambiguously selected.

The foregoing features are of representative embodiments and are presented to assist in understanding the invention. It should be understood that they are not intended to be considered limitations on the invention as defined by the claims, or limitations on equivalents to the claims. Therefore, this summary of features should not be considered dispositive in determining equivalents. Additional features of the invention will become apparent in the following description, from the drawings and from the claims.

### BRIEF DESCRIPTION OF THE DRAWINGS

FIG. 1 is a block diagram of a system for loading timepoints according to an exemplary embodiment of the present invention;

FIG. 2 is a flowchart illustrating a method for loading a timepoint according to an exemplary embodiment of the present invention;

FIG. 3 is a flowchart illustrating a method for loading a timepoint for comparison with a previously loaded timepoint according to an exemplary embodiment of the present invention;

FIG. 4 is a patient browser for selecting a timepoint to be loaded according to an exemplary embodiment of the present invention;

FIG. 5 is a chart illustrating a hierarchy for creating a timepoint according to an exemplary embodiment of the present invention;

FIG. 6 is a series list dialog showing valid and invalid image series of timepoints for loading according to an exemplary embodiment of the present invention;

FIG. 7 is a flowchart illustrating a method for loading timepoints according to another exemplary embodiment of the present invention;

FIG. 8 is a flowchart illustrating a method for ambiguous loading of timepoints according to another exemplary embodiment of the present invention.

### DETAILED DESCRIPTION OF EXEMPLARY EMBODIMENTS

Exemplary embodiments of the present invention are directed to a multi-modality application that allows the comparison of two or more studies to each other. This is typically done by comparing an initial diagnosis with a follow-up scan after treatment. For example, the present invention may be used in oncology cases where one or several follow-up studies are performed to evaluate disease progression and response to therapy. The present invention may also be applied in medical modalities where change detection can be used to detect lesions, tumors, cancers, etc.

For example, the present invention may be used in the following areas of medical imaging: therapy response monitoring by performing change detection using computed tomography (CT) or Magnetic Resonance (MR) images—positron emission tomography (PET) or CT—single photon emission computed tomography (SPECT) over time; bone cancer detection by performing bone segmentation and lesion detection; liver cancer detection using perfusion and spectroscopy; breast cancer detection combining perfusion and spectroscopy and characterizing benign or malignant tumors; and neurology by using semi-automatic and automatic tools for volumetry of brain structures like hippocampal volumes.

The modalities for use with the present invention are, for example: static attenuation corrected (AC) PET, static non-attenuation corrected (NAC) PET and respiratory-gated PET; static AC SPECT or nuclear medicine (NM) and static NAC SPECT or NM; high-resolution CT, low-resolution CT, spiral CT and respiratory-gated CT; high-resolution magnetic resonance (MR); and ultrasound. The present invention may load gantry-titled datasets. In addition, the present invention is capable of accepting an image series containing unequally spaced slices or an image series containing overlapping slices.

The present invention may further load static AC PET or static NAC PET datasets fused together with corresponding registered CT datasets from one patient study, acquired via a PET/CT scanner or on separate devices. In addition, static AC SPECT or static NAC SPECT datasets fused together with corresponding registered CT datasets from one patient study, acquired via a SPECT/CT scanner or on separate devices may be loaded. Further, two series of the same modality type may be loaded and displayed fused within a single timepoint. For example, the present invention may allow a CT dataset fused together with another CT dataset, where both datasets were acquired via the same CT scanner or different devices.

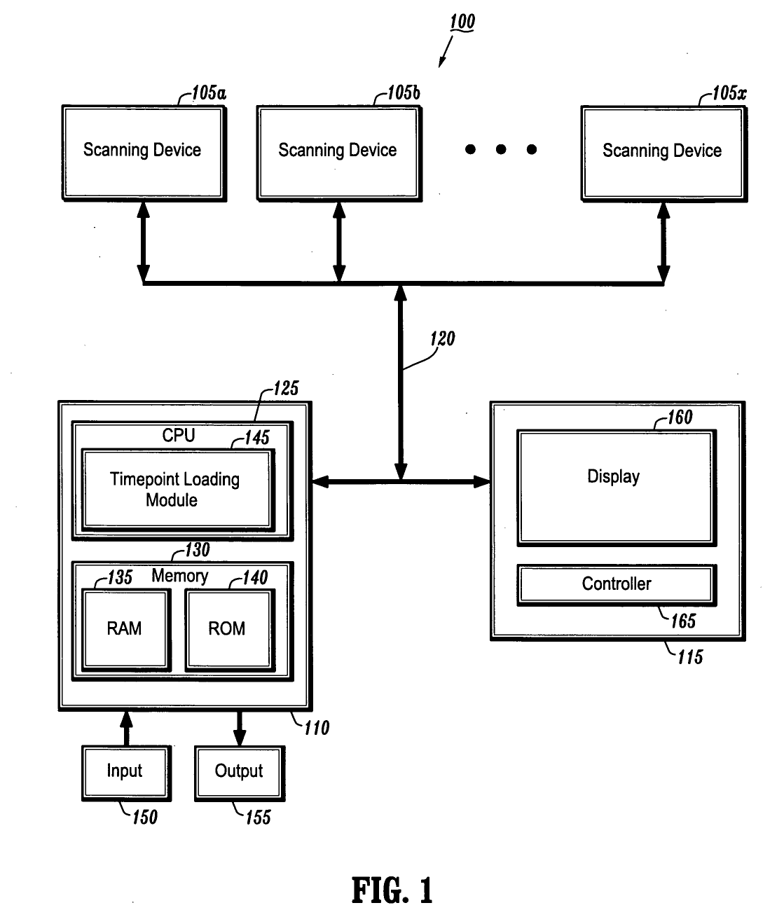

FIG. 1 is a block diagram of a system 100 for loading timepoints for analysis of disease progression or response to therapy according to an exemplary embodiment of the present invention.

As shown in FIG. 1, the system 100 includes, inter alia, several scanning devices 105a, b . . . x, a computer 110 and an operator's console 115 connected over a network 120. The scanning devices 105a, b . . . x may each be one of an MR imaging device, CT imaging device, helical CT device, PET device, SPECT device, hybrid PET-CT device, hybrid SPECT-CT device, two-dimensional (2D) or three-dimensional (3D) fluoroscopic imaging device, 2D, 3D, or four-dimensional (4D) ultrasound imaging device, or an x-ray device. In addition to the aforementioned scanning devices, one or all of the scanning devices 105a, b . . . x may be a multi-modal or hybrid scanning device that is capable of scanning, for example, in a PET mode, SPECT mode or MR mode or generate PET and CT scans from a single hybrid device.

The computer 110, which may be a portable or laptop computer, a personal digital assistant (PDA), etc., includes a central processing unit (CPU) 125 and a memory 130, which are connected to an input 150 and an output 155. The CPU 125 includes a timepoint loading module 145 that includes one or more methods for loading timepoints for analysis of disease progression or response to therapy.

The memory 130 includes a random access memory (RAM) 135 and a read only memory (ROM) 140. The memory 130 can also include a database, CD, DVD, disk drive, etc., or a combination thereof. The RAM 135 functions as a data memory that stores data used during execution of a program in the CPU 125 and is used as a work area. The ROM 140 functions as a program memory for storing a program executed in the CPU 125. The input 150 is constituted by a keyboard, mouse, etc., and the output 155 is constituted by a liquid crystal display (LCD), cathode ray tube (CRT) display, or printer.

The operation of the system 100 is controlled from the operator's console 115, which includes a controller 165, for example, a keyboard, and a display 160, for example, a CRT display. The operator's console 115 communicates with the computer 110 and the scanning device 105 so that 2D image data collected by the scanning devices 105a, b . . . x can be rendered into 3D data by the computer 110 and viewed on the display 160. It is to be understood that the computer 110 can be configured to operate and display information provided by the scanning devices 105a, b . . . x absent the operator's console 115, using, for example, the input 150 and output 155 devices to execute certain tasks performed by the controller 165 and display 160.

The operator's console 115 further includes any suitable image rendering system/tool/application that can process digital image data of an acquired image dataset (or portion thereof) to generate and display 2D and/or 3D images on the display 160. More specifically, the image rendering system may be an application that provides 2D/3D rendering and visualization of medical image data, and which executes on a general purpose or specific computer workstation. The computer 110 may also include an image rendering system/tool/application for processing digital image data of an acquired image dataset to generate and display 2D and/or 3D images.

As shown in FIG. 1, the timepoint loading module 145 may also be used by the computer 110 to receive and process digital medical image data, which as noted above, may be in the form of raw image data, 2D reconstructed data (e.g., axial slices), or 3D reconstructed data such as volumetric image data or multiplanar reformats, or any combination of such formats. The data processing results can be output from the computer 110 via the network 120 to an image rendering system in the operator's console 115 for generating 2D and/or 3D renderings of image data in accordance with the data processing results, such as segmentation of organs or anatomical structures, color or intensity variations, and so forth.

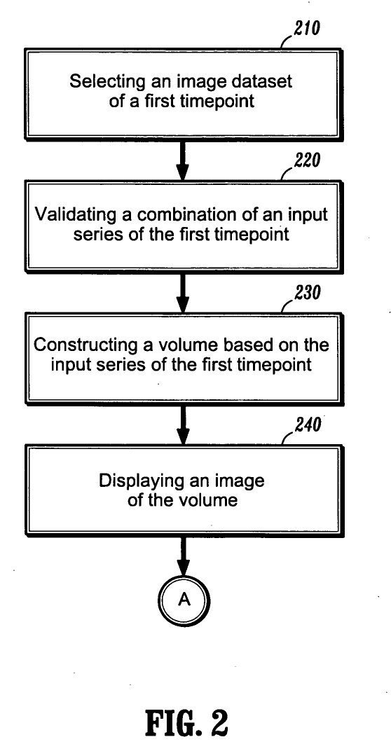

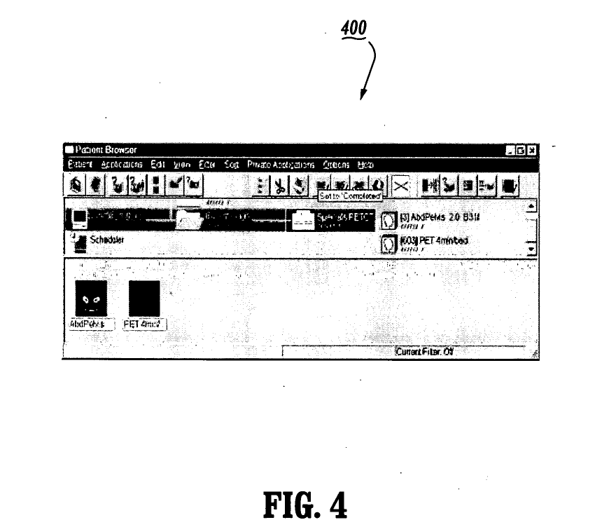

FIG. 2 is a flowchart illustrating a method for loading a timepoint according to an exemplary embodiment of the present invention. As shown in FIG. 2, a user selects an image dataset of a first timepoint via a patient browser 400 of FIG. 4 (210). The image dataset of the first timepoint may include one of the following combinations of image series: a single CT series; a single PET series; a single SPECT series; a combination of a CT and PET series from the same study or from different studies; and a combination of a CT and SPECT series from the same study or from different studies. Exemplary dataset combinations for a single timepoint are listed below in Table 1.

TABLE 1

DATASETS OR COMBINATIONS
FOR A SINGLE TIMEPOINT
A single CT series
A single PET-AC series
A single PET-NAC series
A single SPECT-AC series
A single SPECT-NAC series
CT series + PET-AC series
CT series + PET-NAC series
CT series + SPECT-AC series
CT series + SPECT-NAC series
A single MR series
MR series + PET-AC series
MR series + PET-NAC series
MR series + SPECT-AC series
MR series + SPECT-NAC series

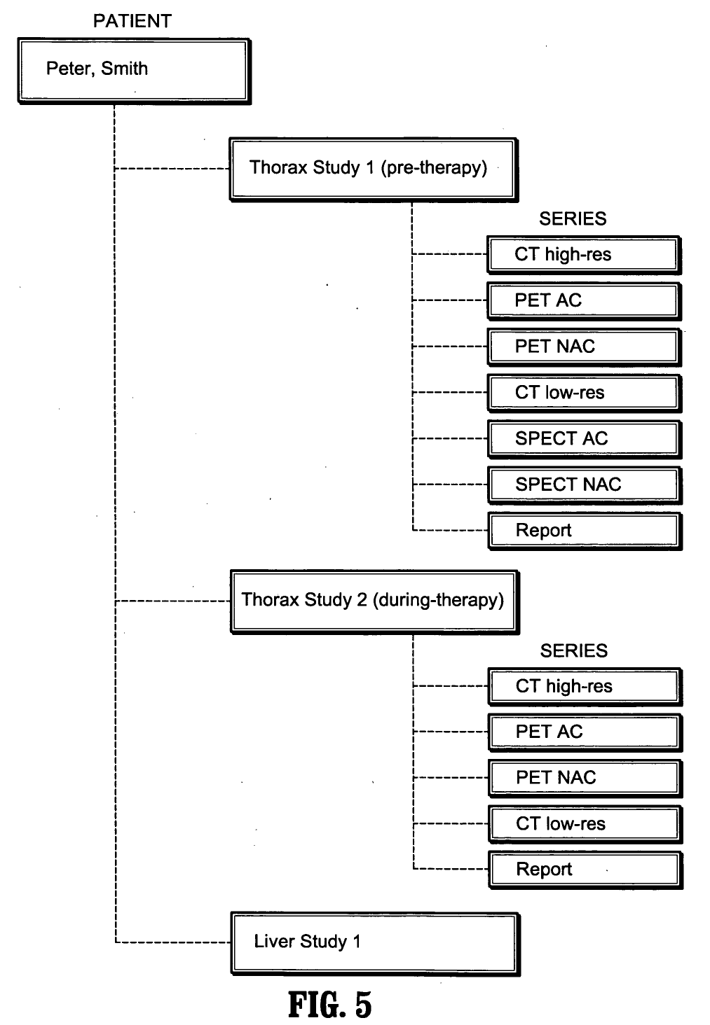

The image datasets of the first timepoint and subsequent timepoints could be from pre-therapy, during-therapy or post-therapy studies. In addition, the same image series can be included as a series in both the first timepoint and subsequent timepoints. For example, in a sample patient hierarchy depicted in FIG. 5, a high-resolution CT series and PET AC series could be combined to form the first timepoint and the high-resolution CT series and a PET NAC series could be combined to form a second timepoint. In other words, a single timepoint could contribute to the first and second timepoints.

Once the image dataset is selected, it can be loaded by dragging and dropping from the patient browser 400 onto a display area; clicking an extension button on the patient browser 400 and double-clicking relevant data on the patient browser 400. For example, a user can perform the relevant selection in the patient browser 400 and click an extension button for loading. The level of selection of the data in the patient browser 400 can be at series, study or at the patient level. In addition, image datasets from sources other than a local database accessible via the patient browser 400 can also be selected for loading. For example, a component, which provides an interface for allowing software packages to integrate, can be user to load from a picture archiving communications systems (PACS) browser.

Subsequent to selecting the image dataset of the first timepoint, the image dataset is validated (220). This is accomplished, for example, by using image header information associated with the image series of the image dataset. Thus, when studies containing different patient header information for single as well as multiple timepoints are selected for loading, a warning dialog may pop-up to indicate to the user that the patient IDs are different and thus indicate the correct manner for loading an image series. The warning dialog may also be used to prompt the user to modify the patient IDs. Once the image dataset has been validated, a volume associated with the image dataset is constructed (230).

The image dataset is constructed by parsing each image and sorting each image based on various image attributes such as scan time, slice position and location. A 3D volume is then constructed based on the image collection. Once the image dataset is constructed, it is displayed on a user interface (240). Now that the image dataset of the first timepoint is loaded, an image dataset of a second timepoint may be loaded.

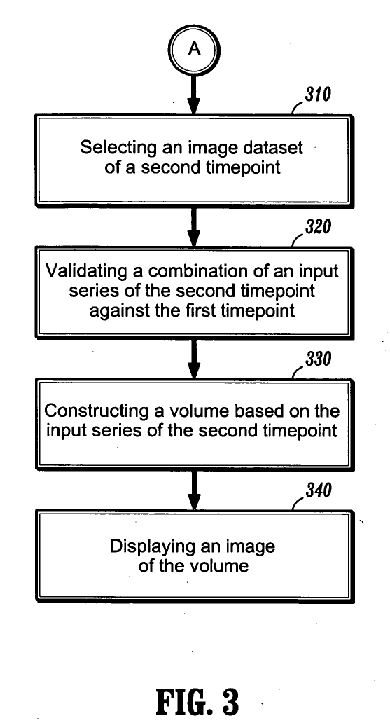

FIG. 3 is a flowchart illustrating a method for loading a timepoint for comparison with a previously loaded timepoint according to an exemplary embodiment of the present invention. As shown in FIG. 3, once the image dataset of the first timepoint is loaded, an image dataset of a second timepoint may be selected (310). Similar to selecting the image dataset of the first timepoint, the image dataset of the second timepoint may be selected via the patient browser 400. In addition, the second timepoint may be one of the image series described above for the first timepoint. After selecting the image dataset of the second timepoint for loading, the image dataset is validated (320).

When validating the image dataset of the second timepoint, it is determined if the image dataset is one of a valid combination of datasets for multiple timepoint loading and then sorted. A list of the valid combinations of datasets for multiple timepoint loading is shown below in Table 2. As shown, for example, in Table 2, if the following combinations are selected: a CT+PET series or CT+SPECT series from one study and a CT+PET series or CT+SPECT series from a different study, they will be loaded automatically.

TABLE 2

| FIRST TIMEPOINT | SECOND TIMEPOINT |
|---|---|
| PET AC alone or with NAC | PET AC alone or with NAC |
| | PET AC alone or with NAC + CT |
| | PET AC alone or with NAC + MR |
| | SPECT |
| SPECT AC alone or with NAC | SPECT AC alone or with NAC |
| | SPECT AC alone or with NAC + CT |
| | SPECT AC alone or with NAC + MR |
| | PET |
| CT | CT |
| | CT + PET AC alone or with NAC |
| | CT + SPECT AC alone or with NAC |
| | MR |
| MR | MR |
| | MR + PET AC alone or with NAC |
| | MR + SPECT AC alone or with NAC |
| | CT |
| PET AC alone or with | PET AC alone or with NAC |
| NAC + CT | CT |
| | PET AC alone or with NAC + CT |
| | MR |
| | SPECT |
| SPECT AC alone or with | SPECT AC alone or with NAC |
| NAC + CT | CT |
| | SPECT AC alone or with NAC + CT |
| | MR |
| | PET |

As further shown in Table 2, if an image dataset of a first timepoint is already loaded containing a SPECT AC dataset alone or with a NAC dataset, any one of the SPECT NAC dataset from the first timepoint, SPECT AC dataset alone or with the NAC dataset and a SPECT AC dataset alone or with an NAC dataset and a CT dataset may be loaded as the second timepoint.

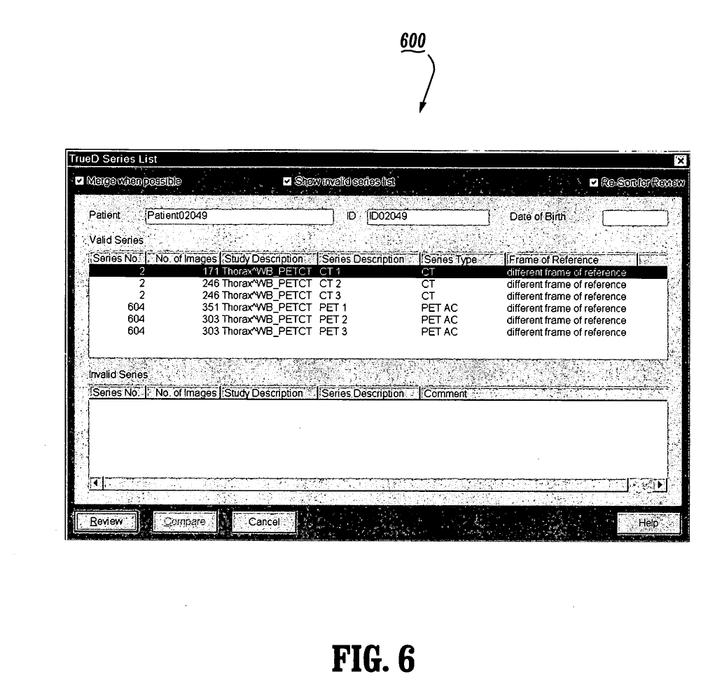

If, however, the image dataset of the second timepoint is not one of the valid combinations of datasets for loading, then a series dialog 600 of FIG. 6 may be displayed indicating valid combinations of datasets for loading to the user. For example, all the series to be loaded could be listed in the series dialog 600 and shown to the user along with a list of invalid series if present. The valid list of series may contain the necessary columns to allow the user to distinguish between the types of series (e.g., modality and NAC/AC columns), and using these, the user may select those series that constitute a valid second timepoint.

As further shown in Table 2, the PET or SPECT AC and NAC datasets are not listed separately because it is assumed that whenever the user selects the PET AC dataset and loads, the PET AC dataset will be displayed. Similarly, when the user selects the PET NAC dataset and loads, the PET NAC dataset will be loaded and displayed along with a CT dataset. The user can then toggle between the PET AC and PET NAC datasets. The same functionality also holds true for the SPECT AC/NAC datasets.

Once the image dataset of the first timepoint has been validated, a volume associated with the image dataset is constructed (330) and then displayed (340). Now that the second timepoint is loaded, it can be compared to the first timepoint. Thus, once the second timepoint is loaded and subsequently displayed, a medical practitioner will be able to compare or diagnose medical conditions or response to therapy across the datasets of the first and second timepoints.

It is to be understood that after the image datasets have been loaded, they are registered. Registration is the process of aligning medical image data. In other words, it is a procedure used to align two input image series generated by different modalities or by one modality at different times. During registration, one of the datasets will be fixed, e.g., in an unchanged position, and the other data set will be transformed, e.g., translated, rotated and scaled to align the two datasets. The fixed dataset may also be referred to as the reference volume and the dataset to be transformed may be referred to as the model volume. Thus, a geometrical transformation is performed for the model volume to match the anatomy of the reference volume.

In accordance with an exemplary embodiment of the present invention, the several registration methods/algorithms may be used. They may be, for example: automatic/mutual information registration (e.g., automatic registration); landmark registration and visual alignment (e.g., manual matching).

Automatic registration is a fully automated matching algorithm based on mutual information or normalized mutual information. Prior to initiating automatic registration, however, the user could perform a visual alignment to improve the performance of the automatic registration. It is to be understood that the automatic registration could occur simultaneous top the step or steps of loading.

Automatic registration comprises the steps of: registering a first image series with a second image series of the first image dataset of the first timepoint; registering the first image series of the first image dataset of the first timepoint with a first image series of the second image dataset of the second timepoint; and registering the first image series of the second image dataset of the second timepoint with a second image series of the second image dataset of the second timepoint.

For example, when two CT-PET scans are loaded, registration of the CT-PET scans begins for both first and second timepoints in sequence. Once the CT-PET registrations are completed, a registration is initiated to match the two CT studies across the first and second timepoints. While the automatic registration takes place, the progress of the registration can be visualized in alpha blended images (e.g., fused images). A progress text may also be displayed that indicates the current progress of the automatic registration.

Landmark registration is the identification of known marks at unisonous positions in both image series. From that identification, the algorithm calculates the registration. Visual alignment is done on a fused dataset. The reference series remains fixed and using visual alignment controls, the model series can be translated/rotated to align with the reference image.

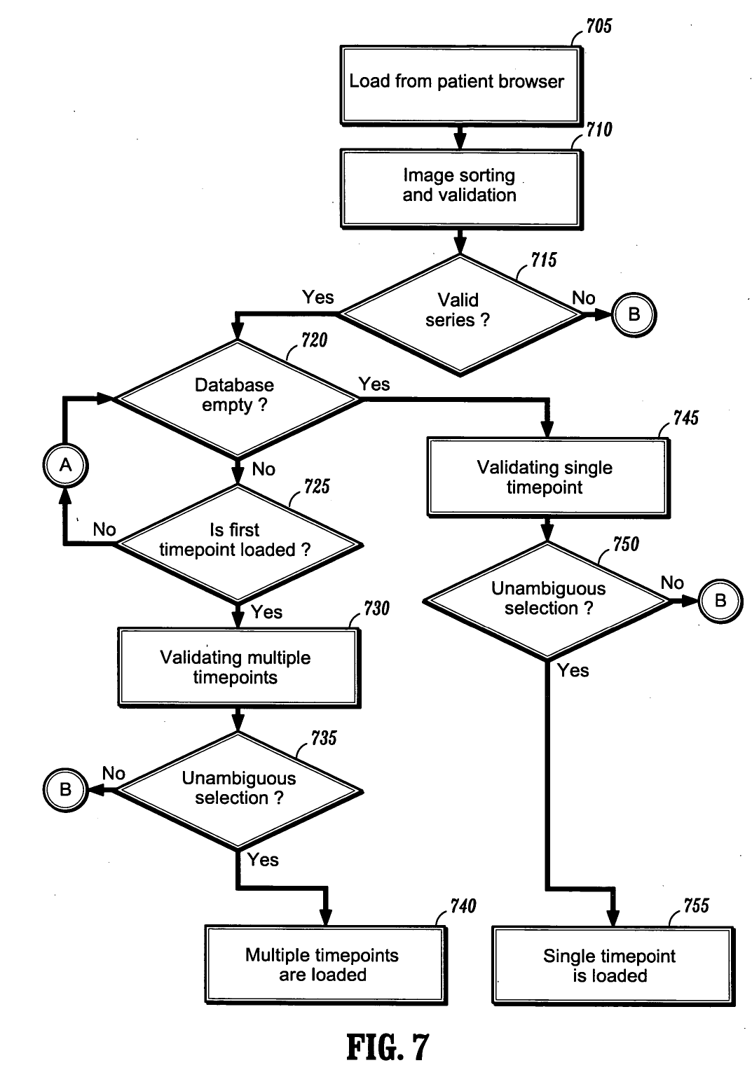

FIG. 7 is a flowchart illustrating a method for loading timepoints according to another exemplary embodiment of the present invention. As shown in FIG. 7, an image dataset of a timepoint is selected for loading from a patient browser (705). The image dataset is then sorted and validated (710). In other words, the image dataset is checked to see whether it will form a suitable 3D volume and then sorted. The image dataset may be validated as described above with reference to FIG. 2. The sorting of the image datasets may be performed by using the series list dialog 600. For example, the series list dialog 600 indicates when an invalid series is present in the image dataset selected for loading, thus enabling a user to reselect a proper series for loading. However, for all the combinations listed above in Table 1, the loading takes place without the display of the series list dialog as these cases are considered to be unambiguous loading scenarios.

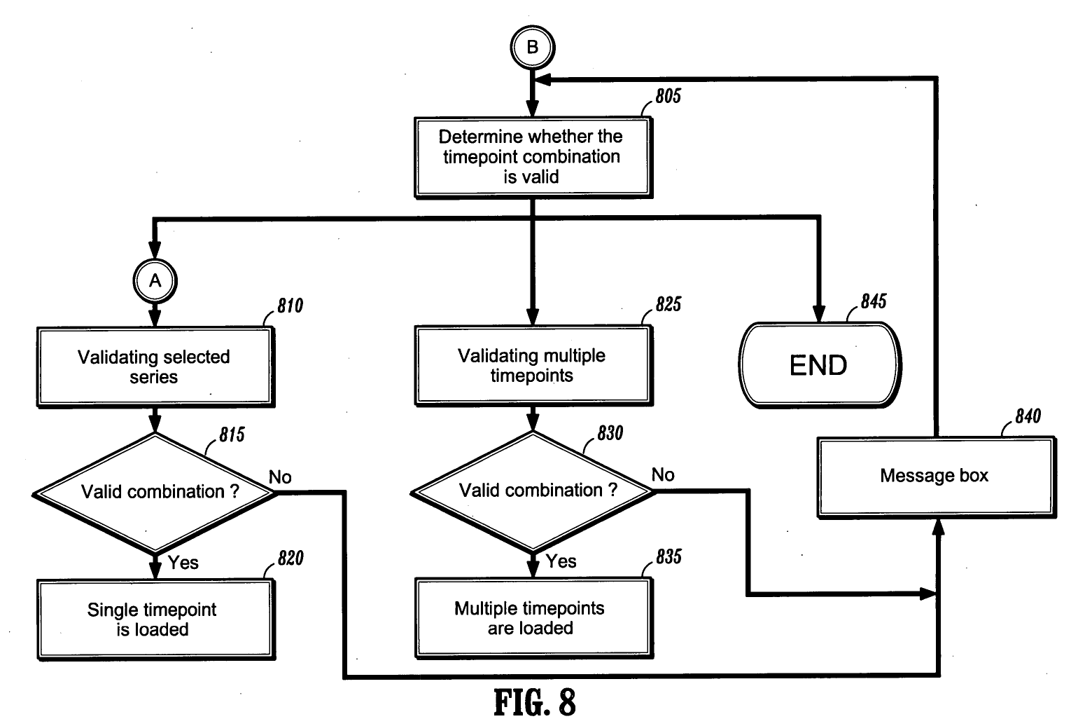

Once the image dataset has been sorted and validated, it is determined if the image dataset of the timepoint is a valid image dataset for loading (715). Thus, if the image dataset was validated in step 710, the process moves on to determine if a timepoint storage database is empty (720). If the image dataset was not validated in step 710, the process moves on to determine if the timepoint was ambiguously selected (B). This will be described in more detail hereinafter with reference to FIG. 8.

If it is determined that the database is not empty in step 720, a timepoint has already been loaded (725). When a timepoint has already been loaded, it is determined if the newly loaded timepoint and the previously loaded timepoint include a valid combination of image datasets for multiple timepoint loading (730). A list of the valid combinations of datasets for multiple timepoint loading is shown above in Table 2.

After validating the combinations of datasets for multiple timepoint loading, it is determined if the image datasets were unambiguously selected (735). The combination of image datasets is determined to be unambiguously selected if it is one of the valid combinations of datasets listed above in Table 2. If the selection is unambiguous, multiple timepoints are loaded (740) and then displayed for comparison. Referring back to step 725, if a timepoint has not already been loaded but the database contains certain data such as two timepoints that were previously loaded, both timepoints will be unloaded and the specified load will be loaded, thus the database is unloaded (A).

If, however, no datasets have been loaded in step 720, the image dataset of the selected timepoint is validated for a single timepoint load (745). The image dataset may be validated for loading if it satisfies one of the conditions listed below in Table 3.

TABLE 3

| DATASETS SELECTED | RESULTANT LOAD |
|---|---|
| All the combinations in Table 1 | The corresponding datasets should be |
| | loaded as a first timepoint |
| A single series having multiple | CT or PET or SPECT images from the |
| volumes (CT and PET or | single series of the specified study |
| SPECT) | should be loaded as a first timepoint |
| Study with single series (CT or | CT or PET or SPECT images from the |
| PET or SPECT) (a) | single series of the specified study |
| | should be loaded as a first timepoint |
| Study with the following | CT high-resolution and PET should be |
| configurations: CT high- | loaded in a fused display as a first |
| resolution, PET AC and/or PET | timepoint |
| NAC (optionally) (b) | |
| Study with the following | CT high-resolution and SPECT should |
| configurations: CT high- | be loaded in a fused display as a first |
| resolution, SPECT AC and/or | timepoint |
| SPECT NAC (optionally) (c) | |
| Patient with study configuration | Directly load the CT or PET or SPECT |
| (a) | images from the single series of the |
| | specified study |
| Patient with study configuration | CT high-resolution and PET should be |
| (b) | loaded in a fused display |
| Patient with study configuration | CT high-resolution and SPECT should |
| (c) | be loaded in a fused display |

If the selected timepoint selection is unambiguous (750), that is, if the image dataset of the timepoint satisfies one of the conditions in Table 3, the timepoint is loaded (755), the loading process is complete and the loaded images may be displayed for comparison. If, however, the timepoint is ambiguous (750), the process proceeds with ambiguous loading (B) as will be described hereinafter with reference to FIG. 8 FIG. 8 is a flowchart illustrating a method for ambiguous loading of timepoints according to another exemplary embodiment of the present invention. As shown in FIG. 8, once it is determined that the selected timepoint selection is ambiguous, the series dialog 600 may be used to aid the user to find a valid timepoint combination (805). The series dialog 600 is typically shown to the user when they load: more than two image datasets; a combination of a SPECT+PET series; a single study having more than two series; multiple studies having multiple series; a patient having multiple studies; or an invalid input series.

When a new timepoint, for example a first timepoint, has been selected from the series dialog and the timepoint storage database contains data, the data is unloaded (A). The timepoint is then validated (810) by determining if it is one of the exemplary dataset combinations for a single timepoint listed above in Table 1. If the timepoint is a valid combination (815) it is loaded (820) and the loaded images may be displayed for comparison. If, however, the timepoint is not a valid combination (815) a message appears in, for example, a pop-up box (840) prompting the user to reselect the input image series from the series dialog 600. At this point, the user may decide not to continue with the process by canceling the display of the series dialog 600 and ending the process (845).

When a new timepoint has been selected using the series dialog 600 with a timepoint having already been loaded, it is determined if the newly selected timepoint and the previously loaded timepoint include a valid combination of image datasets for multiple timepoint loading (830). A list of the valid combinations of image datasets for multiple timepoint loading is shown above in Table 2. If the newly selected timepoint and previously loaded timepoint are a valid combination of datasets for multiple timepoint loading, the newly selected timepoint is loaded (835). If, however, the newly selected timepoint and the previously loaded timepoint are not a valid combination (830) a message appears in a pop-up box (840) prompting the user to reselect the input image series from the series dialog 600. The user may also end the process by canceling the display of the series dialog 600 (845).

According to an exemplary embodiment of the present invention, image datasets belonging to a variety of modalities may be used for diagnosis and reporting purposes. This enables, medical practitioners to efficiently compare patient scans from two different time points (e.g., pre- and post-therapy). By automatically registering and displaying PET/CT or SPECT-CT images from studies acquired at different times, the present invention assists medical practitioners in making better-informed diagnostic, therapy and follow-up decisions.

It is to be understood that the present invention may be implemented in various forms of hardware, software, firmware, special purpose processors, or a combination thereof. In one embodiment, the present invention may be implemented in software as an application program tangibly embodied on a program storage device (e.g., magnetic floppy disk, RAM, CD ROM, DVD, ROM, and flash memory). The application program may be uploaded to, and executed by, a machine comprising any suitable architecture.

It is to be further understood that because some of the constituent system components and method steps depicted in the accompanying figures may be implemented in software, the actual connections between the system components (or the process steps) may differ depending on the manner in which the present invention is programmed. Given the teachings of the present invention provided herein, one of ordinary skill in the art will be able to contemplate these and similar implementations or configurations of the present invention.

It should also be understood that the above description is only representative of illustrative embodiments. For the convenience of the reader, the above description has focused on a representative sample of possible embodiments, a sample that is illustrative of the principles of the invention. The description has not attempted to exhaustively enumerate all possible variations. That alternative embodiments may not have been presented for a specific portion of the invention, or that further undescribed alternatives may be available for a portion, is not to be considered a disclaimer of those alternate embodiments. Other applications and embodiments can be implemented without departing from the spirit and scope of the present invention.

It is therefore intended, that the invention not be limited to the specifically described embodiments, because numerous permutations and combinations of the above and implementations involving non-inventive substitutions for the above can be created, but the invention is to be defined in accordance with the claims that follow. It can be appreciated that many of those undescribed embodiments are within the literal scope of the following claims, and that others are equivalent.

What is claimed is:

1. A method for loading a timepoint for comparison with a previously loaded timepoint, comprising:
selecting an image dataset of the timepoint;
validating the image dataset of the timepoint against a validated image dataset of the previously loaded timepoint; and
constructing a volume based on the image dataset of the timepoint.

2. The method of claim 1, wherein the image dataset of the timepoint and an image dataset of the previously loaded timepoint each comprise data acquired from one of a computed tomography (CT), positron emission tomography (PET), single photon emission computed tomography (SPECT), magnetic resonance (MR) and ultrasound modality.

3. The method of claim 2, wherein the image dataset of the timepoint and the image dataset of the previously loaded timepoint each comprise one of a CT image series and MR image series, a PET image series and SPECT image series, a combination of a CT and PET image series, a combination of an MR and PET image series, a combination of a CT and SPECT image series, a combination of an MR and SPECT image series and an ultrasound image series.

4. The method of claim 3, wherein the image series in each of the image dataset of the timepoint and the image dataset of the previously loaded timepoint comprise data from one of a pre-therapy, ongoing therapy and post-therapy study.

5. The method of claim 1, further comprising:
registering the image dataset of the timepoint and the image dataset of the previously loaded timepoint using one of automatic registration, landmark registration and visual registration.

6. The method of claim 5, wherein automatic registration used during the step of registering the image dataset of the timepoint and the image dataset of the previously loaded timepoint comprises:
registering a first image series with a second image series of the image dataset of the timepoint;
registering the first image series of the image dataset of the timepoint with a first image series of the image dataset of the previously loaded timepoint; and
registering the first image series of the image dataset of the previously loaded timepoint with a second image series of the image dataset of the previously loaded timepoint.

7. A method for loading timepoints for analysis of disease progression or response to therapy, comprising:
selecting an image dataset of a first timepoint;
loading the image dataset of the first timepoint;
validating the image dataset of the first timepoint;
constructing a volume based on the image dataset of the first timepoint;
selecting an image dataset of the second timepoint;
loading the image dataset of the second timepoint;
validating the image dataset of the second timepoint against the validated image dataset of the first timepoint; and
constructing a volume based on the image dataset of the second timepoint.

8. The method of claim 7, wherein the image dataset of the first timepoint and an image dataset of the second timepoint each comprise data acquired from one of a computed tomography (CT), positron emission tomography (PET), single photon emission computed tomography (SPECT), magnetic resonance (MR) and ultrasound modality.

9. The method of claim 8, wherein the image dataset of the first timepoint and the image dataset of the second timepoint each comprise one of a CT image series and MR image series, a PET image series and SPECT image series, a combination of a CT and PET image series, a combination of an MR and PET image series, a combination of a CT and SPECT image series, a combination of an MR and SPECT image series and an ultrasound image series.

10. The method of claim 9, wherein the image series in each of the image dataset of the first timepoint and the image dataset of the second timepoint comprise data from one of a pre-therapy, ongoing therapy and post-therapy study.

11. The method of claim 7, further comprising:
determining whether the image dataset of the first timepoint was ambiguously selected.

12. The method of claim 7, further comprising:
determining whether the image dataset of the second timepoint was ambiguously selected.

13. A system for loading a timepoint for comparison with a previously loaded timepoint, comprising:
a memory device for storing a program;
a processor in communication with the memory device, the processor operative with the program to:
select an image dataset of the timepoint;
validate the image dataset of the timepoint against a validated image dataset of the previously loaded timepoint; and
construct a volume based on the image dataset of the timepoint.

14. The system of claim 13, wherein the image dataset of the timepoint and an image dataset of the previously loaded timepoint each comprise data acquired from one of a computed tomography (CT), positron emission tomography (PET), single photon emission computed tomography (SPECT), magnetic resonance (MR) and ultrasound modality.

15. The system of claim 14, wherein the image dataset of the timepoint and the image dataset of the previously loaded timepoint each comprise one of a CT image series and MR image series, a PET image series and SPECT image series, a combination of a CT and PET image series, a combination of an MR and PET image series, a combination of a CT and SPECT image series, a combination of an MR and SPECT image series and an ultrasound image series.

16. The system of claim 15, wherein the image series in each of the image dataset of the timepoint and the image dataset of the previously loaded timepoint comprise data from one of a pre-therapy, ongoing therapy and post-therapy study.

17. The system of claim 14, wherein the processor is further operative with the program code to:
register the image dataset of the timepoint and the image dataset of the previously loaded timepoint using one of automatic registration, landmark registration and visual registration.

18. The system of claim 17, wherein the processor is further operative with the program code when automatically registering the image dataset of the timepoint and the image dataset of the previously loaded timepoint to:
register a first image series with a second image series of the image dataset of the timepoint;
register the first image series of the image dataset of the timepoint with a first image series of the image dataset of the previously loaded timepoint; and
register the first image series of the image dataset of the previously loaded timepoint with a second image series of the image dataset of the previously loaded timepoint.

19. A system for loading timepoints for analysis of disease progression or response to therapy, comprising:
a memory device for storing a program;
a processor in communication with the memory device, the processor operative with the program to:
select an image dataset of a first timepoint;
load the image dataset of the first timepoint;
validate the image dataset of the first timepoint;
construct a volume based on the image dataset of the first timepoint;
select an image dataset of the second timepoint;
load the image dataset of the second timepoint;
validate the image dataset of the second timepoint against the validated image dataset of the first timepoint; and
construct a volume based on the image dataset of the second timepoint.

20. The system of claim 19, wherein the image dataset of the timepoint and an image dataset of the previously loaded timepoint each comprise data acquired from at least one of a computed tomography (CT), positron emission tomography (PET), single photon emission computed tomography (SPECT), magnetic resonance (MR) and ultrasound modality.

21. The system of claim 20, wherein the image dataset of the first timepoint and the image dataset of the second timepoint each comprise one of a CT image series and MR image series, a PET image series and SPECT image series, a combination of a CT and PET image series, a combination of an MR and PET image series, a combination of a CT and SPECT image series, a combination of an MR and SPECT image series and an ultrasound image series.

22. The system of claim 21, wherein the image series in each of the image dataset of the first timepoint and the image dataset of the second timepoint comprise data from one of a pre-therapy, ongoing therapy and post-therapy study.

23. The system of claim 19, wherein the processor is further operative with the program code to:
determine whether the image dataset of the first timepoint was ambiguously selected.

24. The system of claim 19, wherein the processor is further operative with the program code to:
determine whether the image dataset of the second timepoint was ambiguously selected.
